(19) United States
(12) Patent Application Publication
(10) Pub. No.: US 2011/0073115 A1
(43) Pub. Date: Mar. 31, 2011

(54) TRACHEAL CUFF FOR PROVIDING SEAL WITH REDUCED PRESSURE ON THE TRACHEAL WALLS

(75) Inventors: Lockett E. Wood, Lyons, CO (US); Sarah Hayman, Boulder, CO (US); Susan Roweton, Boulder, CO (US); Lise-Helene Moloney, Middletown, RI (US); Dana Deardorff, Lafayette, CO (US)

(73) Assignee: Nellcor Puritan Bennett LLC, Boulder, CO (US)

(21) Appl. No.: 12/570,928

(22) Filed: Sep. 30, 2009

(51) Int. Cl. A61M 16/04

(52) U.S. Cl. 128/207.15

(57) ABSTRACT

According to various embodiments, a tracheal tube may include a cuff assembly that relies on mechanical pressure rather than inflation pressure to form a seal against a patient's trachea. Such cuff assemblies may include cone or umbrella-shaped structures that may form seals at substantially lower pressures than traditional inflation cuffs. One or more of such cuff assemblies may be used instead of or in addition to an inflation cuff to provide an improved seal.

### BACKGROUND

The present disclosure relates generally to medical devices and, more particularly, to airway devices, such as tracheal tubes.

This section is intended to introduce the reader to aspects of the art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air, food, fluids, or other substances into the patient. For example, tracheal tubes may be used to control the flow of air or other gases through a patient's trachea. Such tracheal tubes may include endotracheal (ET) tubes, tracheotomy tubes, or transtracheal tubes. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

As many patients are intubated for several days, healthcare workers may need to balance achieving a high-quality tracheal seal with possible patient discomfort. For example, if improperly overinflated, the pressure and/or frictional force of certain types of inflated cuffs against the tracheal walls may result in some tracheal tissue damage. While a cuff may be inflated at lower pressure to avoid such damage, this may lower the quality of the cuff's seal against the trachea. Low cuff inflation pressures may also be associated with allowing folds to form in the walls of the cuff that may serve as leak paths for air as well as microbe-laden secretions.

Additionally, the quality of a cuff's seal against the tracheal passageway may suffer over the duration of a patient's intubation time. For example, a seal may be compromised when a patient coughs, which may dislodge the cuff from a sealed position. Further, when the endotracheal tube is jostled during patient transport or medical procedures, the force of the movement may shift the position of the inflatable cuff within the trachea, allowing gaps to form between the cuff and the tracheal walls.

### BRIEF DESCRIPTION OF THE DRAWINGS

Advantages of the disclosure may become apparent upon reading the following detailed description and upon reference to the drawings in which:

FIG. 1 is a partial cutaway view of an exemplary tracheal tube and cuff assembly inserted into a patient's trachea according to certain embodiments;

FIG. 2 is a perspective view of a tracheal tube with a cuff assembly in an expanded configuration;

FIG. 3 is a perspective view of a tracheal tube with a cuff assembly in a retracted configuration according to certain presently contemplated embodiments;

FIG. 4 is a perspective view of a tracheal tube with a cuff assembly with a half-barrel configuration;

FIG. 5 is a perspective view of a tracheal tube with a cuff assembly with an inflatable portion;

FIG. 6 is a perspective view of a tracheal tube with a cuff assembly with an interrupted barrel configuration;

FIG. 7 is a perspective view of a tracheal tube with a cuff assembly with an hourglass configuration; and

FIG. 8 is a perspective view of a tracheal tube with a cuff assembly and a traditional balloon cuff.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A tracheal tube may be used to seal a patient's airway and provide positive pressure to the lungs when properly inserted into a patient's trachea. A high quality seal of a cuff against the tracheal walls may assist in isolating the lower airway and anchoring the tube in place. However, a conforming seal is often difficult to obtain over long-term intubation. Although physicians may attempt to determine the quality of a cuff seal by monitoring inflation pressure, the intracuff pressure may not provide an accurate picture of whether a cuff is overinflated (i.e., whether the cuff may have the potential to cause tracheal tissue damage). Because the intracuff pressure of tracheal cuffs may be influenced by the surrounding airway pressure, the pressure in the cuff may vary over the course of a breath cycle, increasing during inspiration and decreasing during exhalation. Such variability in the cuff pressure may lead to temporary and cyclical overinflation and underinflation in the cuff. Because the pressure is variable, monitoring the pressure at different points in the breath cycle may lead to different pressure measurements. Accordingly, determining whether a cuff is appropriately inflated may be complex.

Provided herein are anchoring cuffs for tracheal tubes that do not rely, or rely less on cuff inflation to achieve anchoring and/or sealing of the airway, particularly after initial placement. Such cuff assemblies may be used instead of or in addition to traditional inflatable balloon cuffs. In some exemplary embodiments, a cuff may be mechanically expanded rather than inflated with a gas or a liquid. For example, a mechanically expanded cuff may form a cone or umbrella-shaped structure when expanded within the trachea. The umbrella structure may form a seal with the tracheal walls with less surface area of contact on the tissue, which may in turn reduce the possibility of tracheal damage associated with improper inflation or positioning of the cuff. Further, because an umbrella cuff structure may rely on mechanical contact rather than inflation pressure to form a seal, the seal may be achieved at substantially lower pressures relative to a traditional cuff. In certain disclosed embodiments, such as those that incorporate a traditional balloon cuff, intracuff pressure of the inflated balloon may be used initially to place and seal the cuff, and may also be relied upon at times thereafter to ensure proper operation, but reliance on intracuff pressure alone is reduced or eliminated by the alternative structures disclosed below. Because the disclosed structures are associated with generally lower sealing pressures, they may improve overall safety for the patient.

The disclosed tracheal tubes, systems, and methods may be used in conjunction with any appropriate medical device, including without limitation a feeding tube, an endotracheal tube, a tracheotomy tube, a circuit, an airway accessory, a connector, an adapter, a filter, a humidifier, a nebulizer, nasal cannula, or a supraglottic mask/tube. The tracheal cuffs of the present techniques may be incorporated into systems that facilitate mechanical ventilation of a patient, such as a ventilator. Such systems may typically include connective tubing, a gas source, a monitor, and/or a controller. The controller may be a digital controller, a computer, an electromechanical programmable controller, or any other control system. Further, the devices and techniques provided herein may be used to intubate a trauma victim, an intubated patient, a patient with a tracheotomy, an anesthetized patient, a cardiac arrest victim, a patient suffering from airway obstruction, or a patient suffering from respiratory failure.

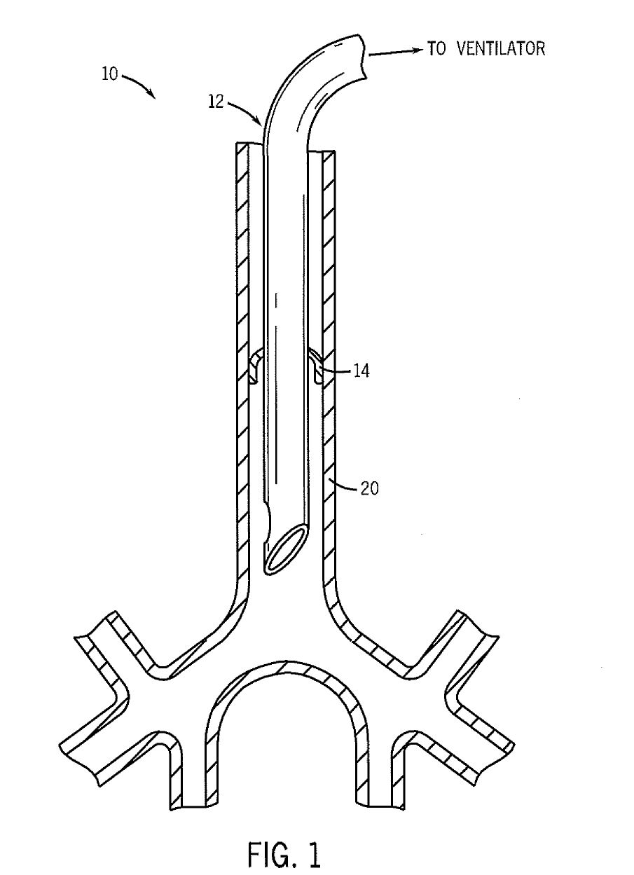

FIG. 1 shows an exemplary tracheal tube system 10 that has been inserted into the trachea of a patient. The system 10 includes a tracheal tube 12, shown here as an endotracheal tube, with a cuff assembly 14 that, as shown, may be mechanically expanded to form a seal against the tracheal walls 20. By relying on mechanical expansion rather than the pressure of a fluid held by an inflated balloon, the pressure exerted on the tracheal walls 20 may be reduced. Typically, balloon cuffs associated with tracheal tubes are inflated within a patient's trachea such that the intracuff pressure is approximately 20-30 cm H₂O. In certain embodiments, cuff assemblies 14 as provided herein may perform adequate tracheal sealing at low pressures. An exemplary cuff assembly 14 may be expanded so that the cuff assembly 14 lightly touches the tracheal walls 28 to initiate and maintain the seal. It is envisioned that a cuff assembly 14 may effectively seal a patient's trachea at exerted pressures of less than 20 cm H₂O, less than 10 cm H₂O or less than 5 cm H₂O.

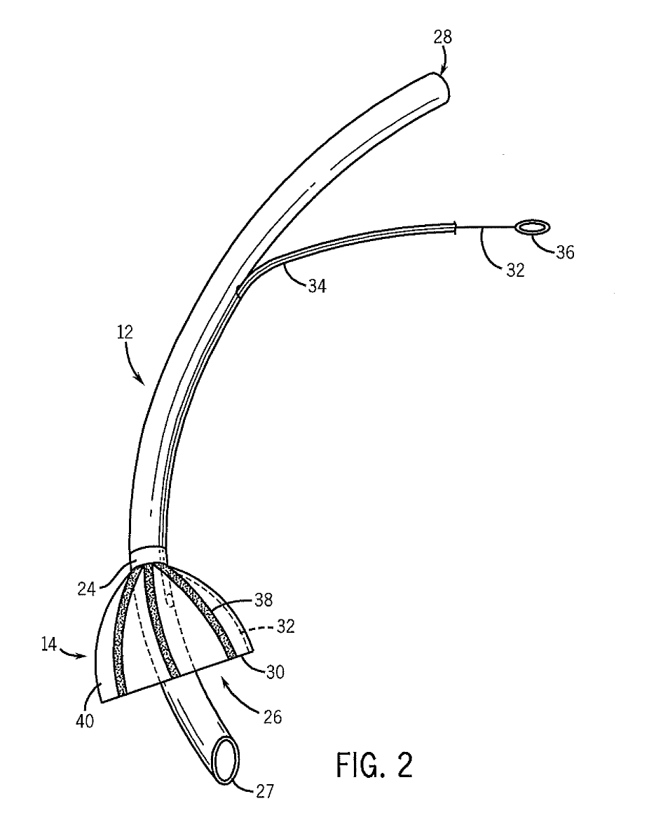

FIG. 2 is a perspective view of the tracheal tube 12 with the cuff assembly 14 in an expanded position, which may correspond to a position for forming a seal with the tracheal walls 20. As shown, the cuff assembly 14 may form an umbrella-shaped structure with one end 24 that is adhered to or otherwise attached to the tracheal tube 12. The other end 26 may be opened, such that the cuff assembly serves to seal the space distal to the cuff assembly 14 from the space proximal to the cuff assembly 14, but does so without being inflated or having a balloon structure. It is envisioned that the cuff assembly 14 may be attached to the tracheal tube 12 in either a convex shape relative to the proximal end 28, as shown in FIG. 2, or a concave shape relative to the proximal end 28. The cuff assembly 14 expands radially outward from the axis of the tube 12.

The tracheal tube 12 may include a mechanism for expanding and retracting the cuff assembly 14. In one embodiment, the cuff assembly 14 may include a channel 30 or opening in the material 40 of the cuff assembly 14 that may accommodate a string, wire, fiber, flexible rod, or similar structure 32. For example, the channel 30 may be formed by a hem (e.g. folding over and attaching an end) of the cuff assembly material 40. Alternatively, the channel may be a separate structure appropriately attached to a surface (e.g., interior or exterior) of the cuff assembly 14. The string 32 may at least partially encircle a circumference of the open end 26 of the cuff assembly 14, such that when the string 32 is pulled, the cuff assembly 14 retracts in a manner similar to a drawstring pouch, but when the string 32 is relaxed, the cuff assembly 14 assumes the expanded position. This expansion as a result of the relaxation of the string 32 may be a result of natural shape memory or rigidity of the material 40 and/or supporting ribs 38. Accordingly, relaxing the string 32 may allow these structures to return to a relaxed position while tightening the string 32 may apply a constricting force on the structures that prevents them from expanding. The string 32 may be threaded through a lumen 34 formed in the walls of the tube 12 that extends outward from the tube 12 so that a pull 36 or tab on the end of string 32 is accessible to an operator when the tube 12 is fully inserted into a patient.

The cuff assembly material 40 may be formed from any material that is may exert sufficient pressure to form a seal against the tracheal walls 20 when in the expanded state, but may also exert low pressures on the tracheal wall 20 (e.g., less than 20-30 cm H₂O) when expanded to form the seal. For example, the cuff assembly material 40 may be a flexible polymer such as polyethylene. In one embodiment, the cuff assembly material 40 may be formed from a shape-shifting polymer or a shape memory material that is configured to change shape upon exposure to a certain temperature, chemical stimulus, or a magnetic field, such as those described in U.S. Pat. Nos. 6,388,043 and 6,720,402, the specifications of which are incorporated by reference in its entirety for all purposes. In one embodiment, the cuff assembly material 40 may be formed from shape-memory alloys, such as NiTi, CuZnAl, and CuAINi alloys.

In another embodiment, the cuff assembly material 40 may be soft and conformable, such as Dow Pellethane® 2363-80A or polyvinyl chloride (PVC). The stiffness of the cuff assembly 14 to form the seal may be provided by support ribs 38 that are formed into, embedded, overmolded by, or otherwise disposed on the cuff assembly material 40 or connecting/between separate panels of cuff assembly material 40. The support ribs 38 may be formed by any suitably stiff material as provided. In embodiments in which the cuff assembly material 40 and/or the support ribs 38 are formed from a material having shape memory, it is envisioned that the shape memory of the cuff assembly 14 may be in the expanded state, such that the default state of the cuff assembly 14 is expanded and force may be exerted to restrain the cuff assembly 14 in the retracted state (such as for intubation and extubation of the patient).

The cuff assembly 14 may also include a mucoadhesive layer that may include a variety of mucoadhesive compositions and/or agents to further seal the cuff assembly 14 to the mucosal tissue of the tracheal walls 20. Suitable mucoadhesives include, but are not limited to hydroxypropyl cellulose, hydroxypropyl methylcellulose, hydroxyethylcellulose, ethylcellulose, carboxymethylcellulose, dextran, cyclodextrins, polysaccharide gums (e.g. guar gum, xanthan gum), polyvinyl pyrrolidone, pectins, starches, collagen, gelatin, alginic acid, hyaluronic acid, fibronectin, casein, acrylic acid polymers, polymers of acrylic acid esters, poly(acrylamide), vinyl polymers, vinyl copolymers, polymers of vinyl alcohols, alkoxy polymers, polyethylene oxide polymers, poly(propylene oxide), poly(propylene glycol), poly(ethylene glycol), poly(methacrylic acid), polyethers, and any combination of the above. Such combinations may include homopolymers and copolymers of the polymers provided as well as mixtures and semi-interpenetrating and interpenetrating networks that include the polymers. In specific embodiments, the mucoadhesive may be a biocompatible polymer, for example polyacrylic acid, that is cross-linked with an acceptable agent to create an insoluble gel. The use of an insoluble gel may provide the advantage of adherence to the mucosal tissue for relatively long periods of time. For patients that experience longer intubation times, mucoadhesives such as cross-linked polyacrylic acid polymers, such as polycarbophils (e.g., Noveon and Carbomer), may be appropriate for use for three to five days or longer. Polycarbophil-based polymers are weak acids and contain many negatively-charged carboxyl-groups. The multiple negative charges on these polymers promote hydrogen-bonding between the polymers and the negatively-charged mucin glycoproteins that mediate attachment of mucus to the epithelial lining. The mucoadhesive may also include chitosan, a deacetylated derivative of chitin, which is a natural biopolymer. A mucoadhesive polymer may also include acrylic acid polymers (e.g. Carbopol® 940, also known as Carbomer® 940, Carbopol 934P and Carbopol® 980, products of BF Goodrich), methyl vinyl/maleic acid copolymers (e.g. Gantrez® S-97, a product of International Specialty Products), polyvinyl pyrrolidone also known as povidone (e.g. Plasdone® K-90, a product of International Specialty Products). These polymers impart relatively high viscosity at relatively low concentrations. They may therefore be incorporated onto the cuff assembly 14 in amounts ranging from about 0.01% to about 10% by weight relative to the total composition. These viscosity-modifying agents further act to improve the film adhesion of the composition to mucous membranes.

Carbopol® 980, in certain embodiments, may be 2-3% by weight of the total composition.

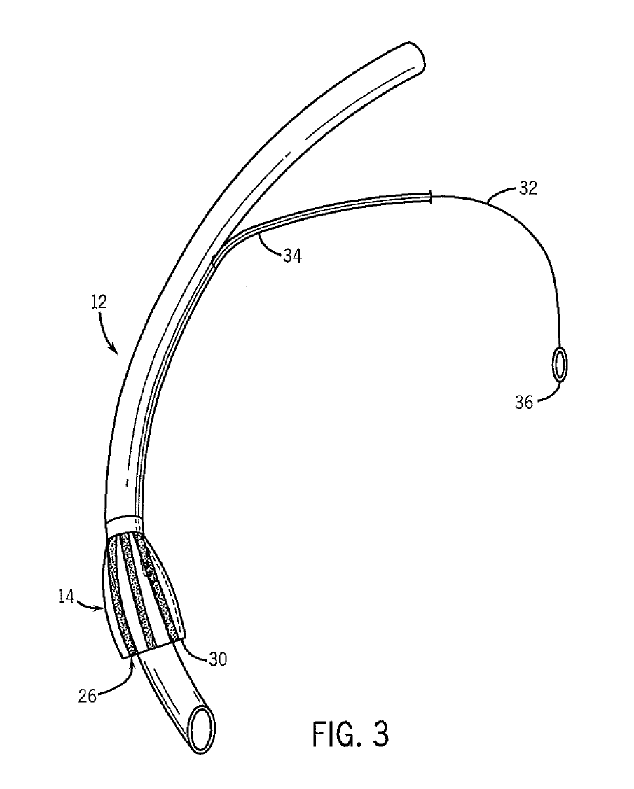

FIG. 3 is a perspective view of the cuff assembly 14 in the retracted position. Retraction may occur by a mechanical process, such as a tightening of a string 32 (or similar structure). For example, if the string 32 is threaded through the channel 30, drawing two ends of the string 32 tight may provide enough restraining force to collapse the material 40 or support ribs 38 in the manner of a drawstring pouch. In an alternative embodiment, the cuff assembly 14 may include a collapsible ring or other structure attached to the material 40 or each of the support ribs 38, for example on the surface opposing the tissue-contacting surface of the cuff assembly 14. The collapsible ring may be coupled to the string 32 such that when the string 32 is tightened, the ring collapses and the force of the ring collapse overcomes the natural rigidity of the material 40 or support ribs 38 and causes the cuff assembly 14 to retract. In another embodiment, a series of strings 32 may be coupled to each support rib 38. The plurality of strings 32 may be attached to pull 36. When pull 36 is actuated to tighten the strings 32, each individual string may pull on its respective support rib 38.

As noted, the material 40 or support ribs 38 may be formed from a material with shape memory. The shape memory may be temperature-sensitive. Accordingly, retraction may take place by exposing the cuff assembly 14 to an appropriate temperature change (e.g., a blast of cold air). Alternatively, a change to a retracted configuration of the shape memory material may be triggered by exposure to a magnetic field or a chemical stimulus. While the retraction may take place through mechanical or other active techniques, it is envisioned that the expandable ribs 38 or material 40 may be formed so that a physician may physically break the seal of the expanded cuff assembly 14 with sufficient force. For example, for a cuff assembly 14 that is convex with respect to the proximal end 28 of the tube 12, just the force of pulling the tube out may cause the cuff assembly 14 to retract sufficiently to allow the seal to break. For a cuff assembly 14 in the opposite orientation, the cuff assembly material 40 may be selected so that the force of a physician actively pulling the tube 12 out of the trachea may break the ribs 38, which may then result in retraction of the cuff assembly 14. In such an embodiment the ribs 38 may be embedded or overmolded within the material 40 so that even upon breaking, no pieces of the ribs 38 would break off of the cuff assembly 14.

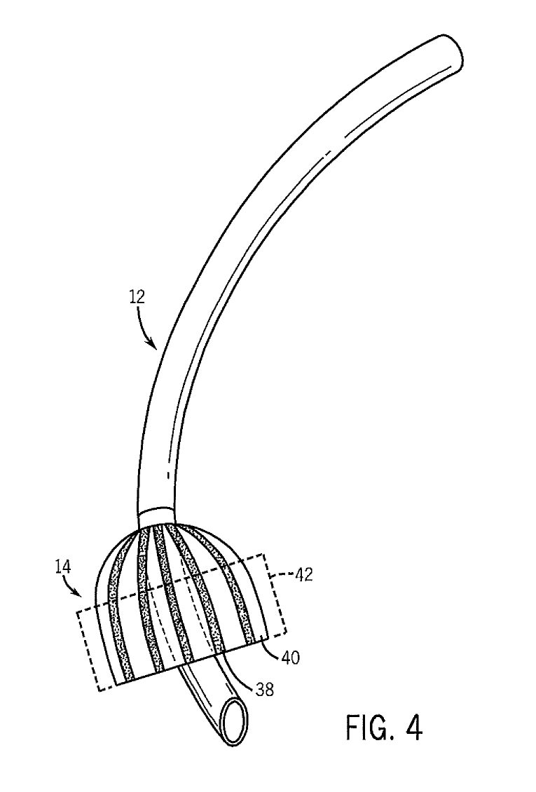

The slope and general shape of the concave or convex cuff assembly 14 may influence the amount of mechanical pressure exerted on the tracheal walls. For example, in addition to more umbrella-shaped structures, the cuff assembly 14 may form a generally half-barrel configuration, shown in FIG. 4, in which the support ribs 38 curve outward but at the point of contact with the tracheal walls 20, are generally straight and elongated. Such a configuration may exert greater total pressure on the tracheal walls relative to an umbrella configuration, but may also serve to dissipate the pressure along a larger surface area, which would lead to lower exerted pressure at any individual point on the tracheal walls 20. In addition, because the seal may be formed with more total surface area of the cuff assembly 14, shown as sealing region 42, certain configurations may provide sealing advantages. Further, cuff assemblies 14 as provided may be configured to have fully expanded diameters that are greater than the average diameter of a patient's trachea (e.g., about 1.5× greater). In such embodiments, the cuff assembly 14 may not be able to fully expand within the trachea, which may provide the ongoing pressure to form the seal as the cuff assembly pushes on the tracheal walls 20. In addition, for cuff assemblies 14 that are in the convex orientation with respect to the proximal end 28 of the tube 12 (e.g., with the open end 26 facing a distal end 27 of the tube 12), the pressure of the cuff assembly 14 may change with the airway pressure changes. For example, the pressure increase in the lower airway during inspiration may cause the cuff assembly 14 to exert greater pressure on the walls of the trachea by pushing on the structure from the underside of the cuff assembly 14. In contrast, for a cuff assembly 14 in the opposite orientation (e.g., with an open end 26 facing a proximal end 28 of the tube 12), increased pressure during inspiration may be met with resistance from the overall stiffness of the cuff assembly 14. In such an orientation, the pressure on the tracheal walls 20 may remain substantially the same or may slightly decrease with increased pressure in the lower airway space.

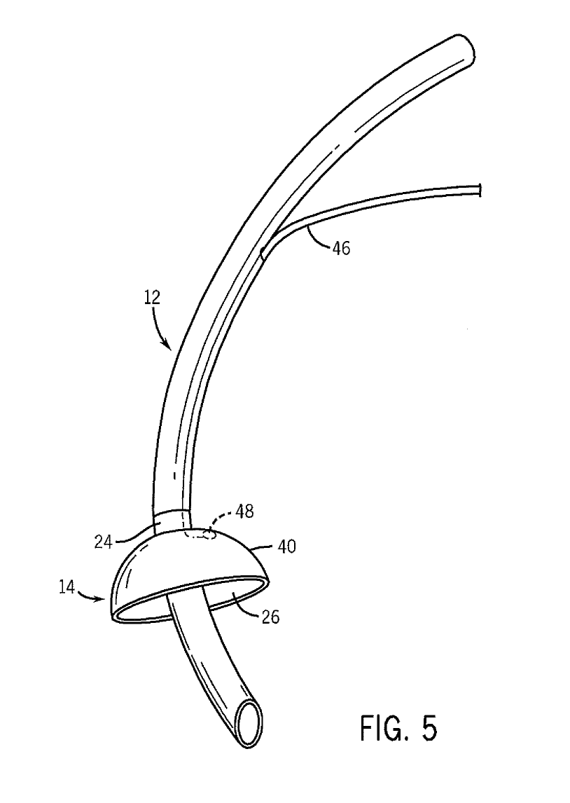

While the previously disclosed embodiments exert mechanical pressure rather than inflation pressure (e.g., they do not include components that are inflatable or that trap air in a fully enclosed structure), in an alternative embodiment, the cuff assembly 14 may include inflatable components that, when inflated, form the umbrella-shaped cuff assembly 14. FIG. 5 is a perspective view of an alternative cuff assembly 14 that may be inflated to form a convex or concave structure. In contrast to inflatable balloon cuffs, the cuff assembly 14 as depicted maintains an open end 26. The inflatable portion of the cuff assembly 14 merely provides stiffness to the cuff assembly material 40. Having inflatable components may allow ease of expansion and retraction, as the cuff assembly 14 may be inflated (e.g., filled with any suitable fluid, such as air, liquid, or an inflatable foam) or deflated by lumen 46 terminating in opening 48 in fluid communication with the inflatable portion of cuff assembly 14, which may be either located on a portion of the tube 12 between sheets of the cuff assembly material 40, or may extend into the inflatable space of the cuff assembly 14. The lumen 46 may extend outside of the tube 12 so that an end is accessible to an operator for inflating or deflating the cuff assembly. In an alternative embodiment, a cuff assembly 14 may include inflatable support ribs 38 to provide stiffness to form the seal. In such an embodiment, the ribs 38 may be inflated via one or more inflation lumens 46. While the depicted assembly 14 may rely at least in part on inflation pressure to form a seal, the pressure against the tracheal walls may be reduced due to the shape of the structure and the reduced volume of trapped fluid inside the cuff assembly as compared to the total surface area.

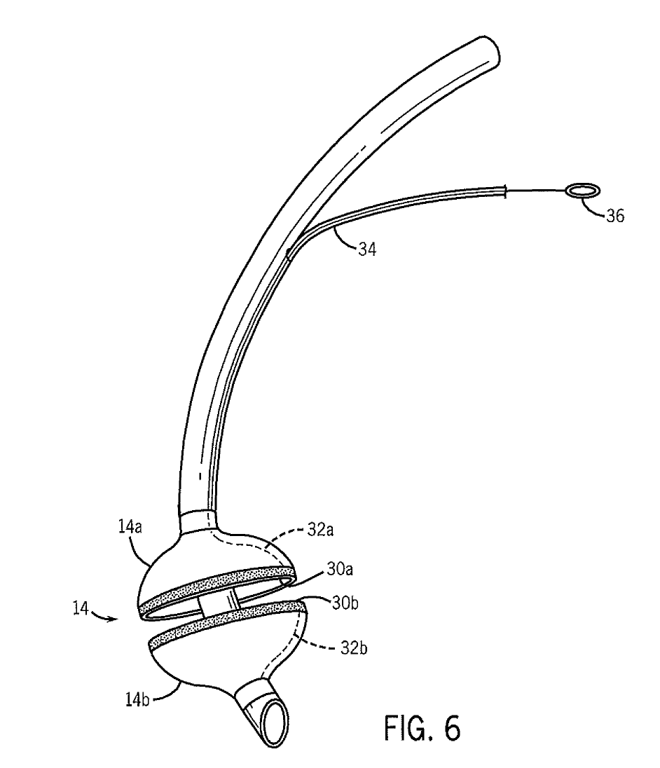

In certain embodiments, a cuff assembly may include both concave and convex structures. FIG. 6 depicts a cuff assembly 14 that includes a convex cuff assembly 14a and a concave cuff assembly 14b relative to the proximal end 28. Such a dual-coned configuration may result in greater surface area of the cuff assembly 14 in contact with the tracheal walls 20. Indeed, such an assembly, by forming an interrupted barrel shape that is similar to the shape of an inflated balloon cuff, may simulate the sealing surface area of a traditional balloon cuff, but with lower pressures associated with mechanical pressure rather than inflation pressure. The individual cuff assembly structures 14a and 14b may be expanded and retracted by any suitable method. As shown, each cuff assembly 14 may include a separate drawstring 32 threaded through one or more lumens 34 that may be drawn within channel 30 by tightening the string with pull 36. The cuff assembly structure 14b forms a bowl that may trap secretions. Such an arrangement may obstruct the flow of secretions into the lungs, where they may cause clinical complications. The cuff assembly 14 may include a suction lumen that terminates in an opening within the bowl that may allow clinicians to suction any accumulated secretions.

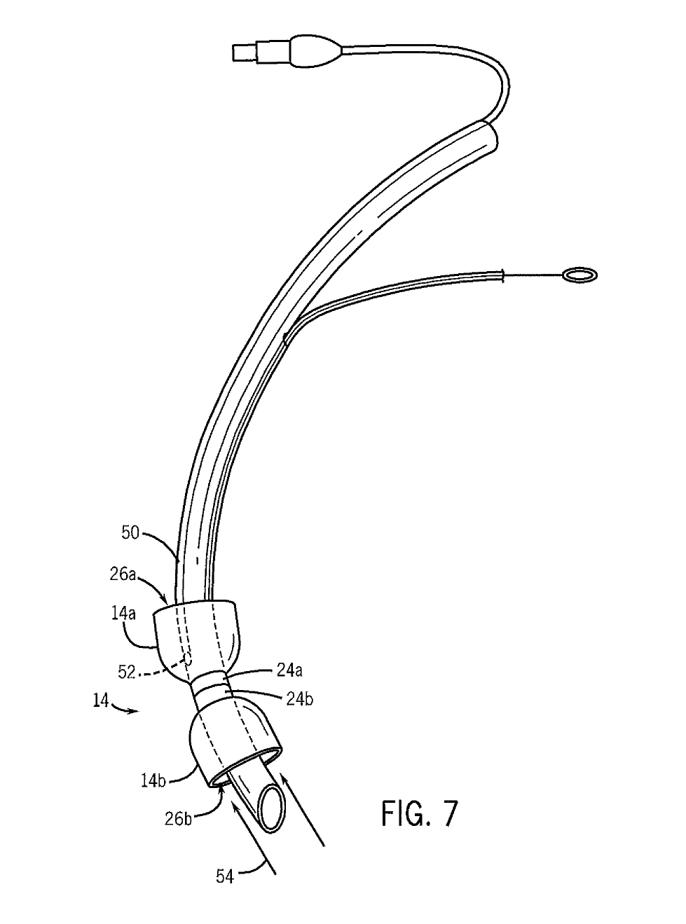

Instead of the depicted interrupted barrel arrangement, in an alternative configuration, the cuff assembly 14 may form an hourglass configuration, as shown in FIG. 7. In such a configuration, an open end of cone 14b may face the trapped airway space (represented by arrows 54) of the distal end 27 of the tube 12. In such an arrangement, the attached ends 24a and 24b may be attached at the same location on the tube 12. The tube 12 may also include a suction lumen 50 that terminates in opening 52 for suctioning any accumulated secretions from the bowl formed by structure 14a.

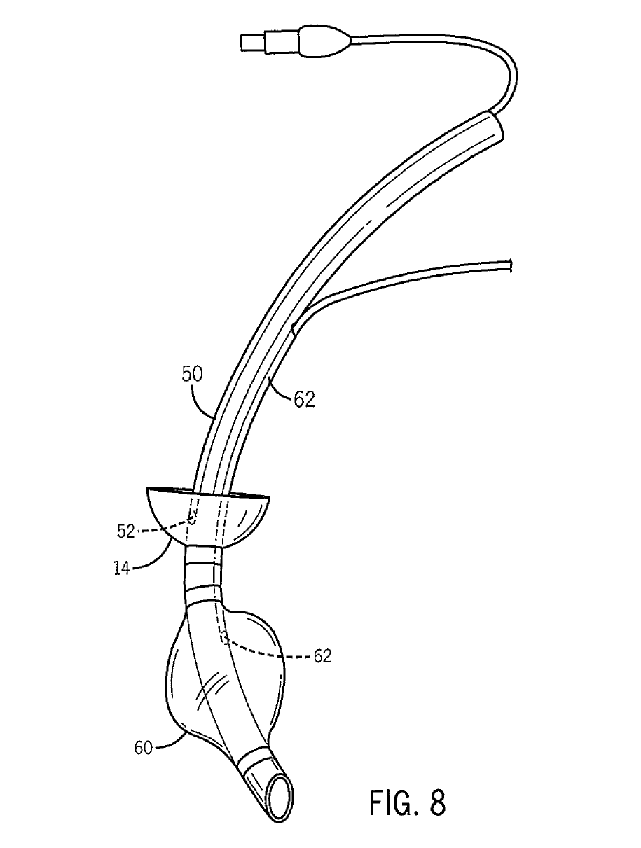

The cuff assemblies 14 may also provide certain benefits when used in conjunction with a traditional inflatable cuff. As shown in FIG. 8, a concave cuff assembly 14 relative to the proximal end 28 may be located proximally to a traditional balloon cuff 60 that is inflated via inflation lumen 62 that terminates in opening 64. The cuff assembly 14 forms a bowl that may trap secretions before they encounter any leak paths present on balloon 60. Suction lumen 50 that terminates in opening 52 within the bowl may allow clinicians to suction any accumulated secretions.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A medical device comprising:
a cuff associated with a tracheal tube, wherein the cuff is configured to expand radially outward from an axis of the tracheal tube and wherein the cuff comprises no fully enclosed spaces capable of retaining a fluid.

2. The medical device, as set forth in claim 1, wherein the cuff comprises an umbrella-shaped structure.

3. The medical device, as set forth in claim 1, wherein the cuff comprises retractable rib members capable of applying pressure to a patient's tracheal walls.

4. The medical device, as set forth in claim 3, wherein the cuff comprises a conformable material in regions connecting or between the retractable rib members.

5. The medical device, as set forth in claim 4, wherein the conformable material comprises polyethylene teraphthalate (PETP), low-density polyethylene (LDPE), polyvinyl chloride (PVC), silicone, neoprene, polyisoprene, or polyurethane (PU)

6. The medical device, as set forth in claim 3, wherein the retractable rib members are formed of a material with shape memory.

7. The medical device, as set forth in claim 6, wherein the material with shape memory returns to a previous shape in response to a temperature change or exposure to a magnetic field.

8. The medical device, as set forth in claim 1, wherein the cuff is configured to assume an expanded position in the absence of a restraining force.

9. The medical device, as set forth in claim 1, comprising a structure associated with the cuff, wherein the structure is adapted to facilitate expansion or collapse of the cuff.

10. The medical device, as set forth in claim 9, wherein the structure comprises a string and wherein tightening of the string facilitates collapse of the cuff.

11. The medical device, as set forth in claim 10, wherein the string is coupled to a second structure, and wherein tightening the string facilitates collapse of the second structure to collapse the cuff.

12. The medical device, as set forth in claim 1, comprising a ventilator to which the tracheal tube is operatively connected.

13. The medical device, as set forth in claim 1, wherein the cuff forms a concave surface with respect to a proximal end of the tracheal tube.

14. A medical device comprising:
a structure configured to expand radially outwardly from an associated tracheal tube to form a substantially concave or convex shape, wherein the concave or convex shape is configured to be open at an end not in contact with the tracheal tube when the structure is expanded.

15. The medical device, as set forth in claim 14, wherein the structure is configured to apply a pressure of less than about 20 cm H₂0 to a patient's tracheal walls when the tracheal tube is inserted into a patient and the structure is expanded radially outward.

16. The medical device, as set forth in claim 14, wherein the structure is configured to apply a pressure of less than about 5 cm H₂0 to a patient's tracheal walls when the tracheal tube is inserted into a patient and the structure is expanded radially outward.

17. The medical device, as set forth in claim 14, wherein the structure comprises one or more inflatable regions configured to form the concave or convex shape when at least partially inflated.

18. The medical device, as set forth in claim 17, wherein the one or more inflatable regions comprise rib members that are connected by a conformable material.

19. The medical device, as set forth in claim 14, comprising a mucoadhesive disposed on at least part of a tissue-contacting surface of the structure.

20. The medical device, as set forth in claim 14, comprising a lumen for suctioning secretions, wherein the lumen comprises an opening in the tracheal tube disposed proximate to the structure.

21. A medical device comprising:
a structure configured to expand radially outwardly from an associated tracheal tube to form a substantially concave or convex shape; and
a balloon cuff associated with the tracheal tube, wherein the balloon cuff is disposed on the tracheal tube distally relative to the structure.

22. The medical device, as set forth in claim 21, wherein the structure forms a concave surface with respect to a proximal end of the tracheal tube.

23. The medical device, as set forth in claim 21, comprising a lumen for suctioning secretions, wherein the lumen comprises an opening in the tracheal tube disposed proximate to the structure.
